(19) United States
(12) Patent Application Publication
(10) Pub. No.: US 2026/0045362 A1
(43) Pub. Date: Feb. 12, 2026

(54) SYSTEMS AND METHODS FOR SELECTION OF PRIORITY-WISE ARTIFICIALLY INTELLIGENT MECHANISMS PER ONE OR MORE CHARACTERISTICS

(71) Applicant: DeepTek Inc., Lewes, DE (US)

(72) Inventors: Viraj Kulkarni, Pune (IN); Richa Pant, Pune (IN); Jitesh Seth, Pune (IN); Ishan Kawathekar, Pune (IN); Ajit Patil, Pune (IN); Amit Kharat, Pune (IN); Aalhad Patwardhan, Pune (IN)

(21) Appl. No.: 19/302,659

(22) Filed: Aug. 18, 2025

Related U.S. Application Data
(63) Continuation of application No. PCT/IB2024/051442, filed on Feb. 15, 2024.

(30) Foreign Application Priority Data
Feb. 16, 2023 (IN) 202321010405

(51) Int. Cl. G16H 50/20

(52) U.S. Cl. CPC G16H 50/20

(57) ABSTRACT

A method for selection of priority-wise artificially intelligent mechanisms per one or more characteristics, said method comprising: receiving (DIM) images as data items (DI); identifying (IDR) an artificially intelligent system (AI1, AI2, AI3, . . . , AIn) used for determination of efficacy, each of the data items (DI), being processed by one or more identified artificially intelligent systems; parsing (SP, PRP), and outputting, scan characteristics and patient characteristics, from the data items (DI), output of said parsing (SP, PRP) being first output (scan characteristics) (O1) and second output (patient characteristics) (O2); analysing (AE) to receive a first output and/or a second output and to receive feedback signal from a feedback model (FM1, FM2, FM3); and serving, as an output (OM), upon analysing (AE), a selection (S) of a priority-wise-ranked artificially intelligent system, said selected system being per parsed scan characteristic (O1) and/or per parsed patient characteristic (O2).

### RELATED APPLICATIONS

This application claims priority under 35 U.S.C. § 120 to, and is a continuation of, co-pending International Application PCT/IB2024/051442, filed Feb. 15, 2024 and designating the US, which claims priority to IN Application 202321010405, filed Feb. 16, 2023, such IN Application also being claimed priority to under 35 U.S.C. § 119. These IN and International applications are incorporated by reference herein in their entireties.

### FIELD

This invention relates to the field of networking systems, computations systems, communication systems, and information systems.

Particularly, this invention relates to the field of healthcare technology, healthcare management, electronic medical records, electronic health records, decision support systems, healthcare information, healthcare reporting, and doctor-patient-interaction systems.

Specifically, this invention relates to systems and methods for selection of priority-wise artificially intelligent mechanisms per one or more characteristics.

### BACKGROUND

Radiology is a medical discipline that uses medical imaging technologies to diagnose diseases. However, the diagnosis of one radiologist may differ from that of another radiologist.

Furthermore, even trained radiologists may overlook some critical findings.

Artificial intelligence (AI) can help overcome subjectivity and improve disease detection accuracy. In recent years, AI algorithms have made substantial advances in image recognition tasks. AI-enabled medical imaging solutions, provided by various AI vendors, enable radiologists to perform accurate and accessible disease screening.

AI systems can be deployed by hospitals, and they aid radiologists by highlighting suspicious regions of interest within the scans. Trained physicians and radiologists can interpret the scan by assessing the AI-generated outputs and reporting findings associated with the scan.

Although there is tremendous interest in employing AI solutions in medical image diagnosis, there is often apprehension when it comes to actually integrating them into clinical practices. Lack of trust, and a dissatisfying user experience, are some of the reasons for slow adoption of AI in clinical practices. Radiologists are, often, uncertain how much trust they should put in AI predictions. The successful implementation of AI in radiology practices ultimately depends on the trust of radiologists in AI outputs.

Therefore, there is a need for systems and methods which solve these aforementioned problems.

### SUMMARY

An object of the invention is to determine efficiency/efficacy/trust quotient of Artificially Intelligent mechanisms.

Another object of the invention is to improve reliability of, and reliance on, Artificially Intelligent mechanisms.

Yet another object of the invention is to improve adoption of Artificially Intelligent mechanisms.

According to this invention, there are provided systems and methods for selection of priority-wise artificially intelligent mechanisms per one or more characteristics, said method comprising the steps of:
receiving images as data items;
identifying an artificially intelligent system being used for determination of efficacy, each of the data items, being processed by one or more identified artificially intelligent systems;
parsing, and outputting, scan characteristics and patient characteristics, from the data items, output of said parsing being first output (scan characteristics) and second output (patient characteristics);
analysing to receive at least a first output and/or at least a second output and configured to receive feedback signal from a feedback model; and
serving, as an output, upon analysing, a selection of a priority-wise-ranked artificially intelligent system, from the plurality of artificially intelligent systems, said selected system being per parsed scan characteristic and/or per parsed patient characteristic.

In at least an embodiment, said paring being selected from a group of parsing steps consisting of:
parsing, and outputting, scan characteristics, from the data items, output of said scan parser being a first output (scan characteristics); and
parsing, and outputting, patient characteristics, from the data items, output of said patient parser being a second output (patient characteristics);

In at least an embodiment, said feedback signal being received from a first feedback module, per artificially intelligent system, in that, feedback relating to accuracy metrics of analysis, correlative to performance metrics of the selected artificially intelligent system, is recorded and fed back to the step of analysing.

In at least an embodiment, said feedback signal being received from a second feedback module, per artificially intelligent system, in that, feedback correlative to first output of the selected artificially intelligent system, is recorded and fed back to the step of analysing.

In at least an embodiment, said feedback signal being received from a third feedback module, per artificially intelligent system, in that, feedback correlative to second output of the selected artificially intelligent system, is recorded and fed back to the step of analysing.

In at least an embodiment, said step of analysing being a cohort-based vectorization analysis configured to determine, and form, cohort-based data sets in order to determine, and form, cohort-based characteristics, characterized, in that,
sorting data items, basis its data and/or its metadata, into cohorts according to sorting rules, correlative to said first output and/or said second output, defined in a sorting rule engine;
using, by a performance processor, one or more data items, with specific metadata, as sorted by said sorter, in order to compute a performance function for all data items belonging to individual cohorts and intersections of two or more cohorts; and
configured to extracting features from the cohorts, upon which a performance function is used by said performance processor, said performance function being correlative to one or more metrics relating to said first output, said performance function being applied to each cohort in order to obtain its feature, said feature processor being configured to collate a set of obtained features, per cohort, to form a feature vector X, for each data item, each vector being shaped by a first set (“k”) of data items and a second set (“n”) of features;

In at least an embodiment, said analysis engine being a cohort-based vectorization module configured to perform the steps of:
receiving the first output and/or the second output and a feedback signal from a feedback model;
computing cohorts based on sorting rules defined in a sorting rule engine, followed by grouping data items with common characteristics;
processing cohorts, using a performance processor, to compute performance metrics for individual cohorts and their intersections;
developing feature vectors based on performance metrics, for data items, sharing common characteristics;
setting a ground truth for training artificially intelligent system using manual mechanisms or machine-fed mechanisms;
calculating trust scores for data items using regression or classification models with weighted features, minimizing a loss function; and
determining prioritization of artificially intelligent system based on threshold values and a rule engine considering the relative importance of characteristics identified by machine learning models.

In at least an embodiment, said step of analysing cooperating with:
generating data, from said first feedback module, correlative to said data items;
outputting said first output from said data items configured to be analysed by said performance parser;
outputting said second output from said data items configured to be analysed by said performance parser; and
establishing Ground Truth data for each cohort, in order to obtain metrics per cohort per data item to be fed to a training module; and
comparing said established ground truth per data item with its own analysed output per data item in order to determine updateable weights based on agreement between said ground truth and said analysed output.

In at least an embodiment, said step of analysing cooperating with:
organizing output of said analysis engine in correlation with said first output and said second output; and
generating a mapping between each considered artificially intelligent system and its corresponding trust score.

In at least an embodiment, said method comprising the steps of:
gathering data items, including ground truth predictions, per data item, made by one or more of the existing artificially intelligent systems, first output, and said second output; and
using said gathered data items, their ground truth predictions, in order to feed to a Training Module which trains internal weights (W) for said analysis engine.

In at least an embodiment, a training module is active only when a ranking module is inactive.

According to this invention, there are provided systems and methods for selection of priority-wise artificially intelligent mechanisms per one or more characteristics, said system comprising:
a data input module configured to receive images, as data items;
an identifier configured to identify an artificially intelligent system being used for determination of efficacy, each of the data items, from the data input module, being processed by one or more identified artificially intelligent systems;
a set of parsers, configured to parse, and output, scan characteristics and patient characteristics, from the data items, output of said parsers being first output (scan characteristics) and second output (patient characteristics);
an analysis engine configured to receive at least a first output and/or at least a second output and configured to receive feedback signal from a feedback model; and
an output module, cooperating with the analysis engine, serving a selection of a priority-wise-ranked artificially intelligent system, from the plurality of artificially intelligent systems, said selected system being per parsed scan characteristic and/or per parsed patient characteristic.

In at least an embodiment, said set of parsers being selected from a group of parsers consisting of:
a scan parser configured to parse, and output, scan characteristics, from the data items, output of said scan parser being a first output (scan characteristics); and
a patient parser configured to parse, and output, patient characteristics, from the data items, output of said patient parser being a second output (patient characteristics);
In at least an embodiment, said feedback model comprising a first feedback module, per artificially intelligent system, in that, feedback relating to accuracy metrics of analysis, correlative to performance metrics of the selected artificially intelligent system, is recorded and fed back to the analysis engine.

In at least an embodiment, said feedback model comprising a second feedback module, per artificially intelligent system, in that, feedback correlative to first output of the selected artificially intelligent system, is recorded and fed back to the analysis engine.

In at least an embodiment, said feedback model comprising a third feedback module, per artificially intelligent system, in that, feedback correlative to second output of the selected artificially intelligent system, is recorded and fed back to the analysis engine.

In at least an embodiment, said analysis engine being a cohort-based vectorization module configured to determine, and form, cohort-based data sets in order to determine, and form, cohort-based characteristics, characterized, in that,
a sorter sorts data items, basis its data and/or its metadata, into cohorts according to sorting rules, correlative to said first output and/or said second output, defined in a sorting rule engine;
a performance processor configured to use one or more data items, with specific metadata, as sorted by said sorter, in order to computes a performance function for all data items belonging to individual cohorts and intersections of two or more cohorts; and
a feature processor configured to extract features from the cohorts, upon which a performance function is used by said performance processor, said performance function being correlative to one or more metrics relating to said first output, said performance function being applied to each cohort in order to obtain its feature, said feature processor being configured to collate a set of obtained features, per cohort, to form a feature vector X, for each data item, each vector being shaped by a first set (“k”) of data items and a second set (“n”) of features;

In at least an embodiment, said analysis engine being a cohort-based vectorization module configured to perform the steps of:
receiving the first output and/or the second output and a feedback signal from a feedback model;
computing cohorts based on sorting rules defined in a sorting rule engine, followed by grouping data items with common characteristics;
processing cohorts, using a performance processor, to compute performance metrics for individual cohorts and their intersections;
developing feature vectors based on performance metrics, for data items, sharing common characteristics;
setting a ground truth for training artificially intelligent system using manual mechanisms or machine-fed mechanisms;
calculating trust scores for data items using regression or classification models with weighted features, minimizing a loss function; and
determining prioritization of artificially intelligent system based on threshold values and a rule engine considering the relative importance of characteristics identified by machine learning models.

In at least an embodiment, said analysis engine cooperating with:
a training module for generating data, from said first feedback module, correlative to said data items;
a scan parser, from said set of parsers, in order to output said first output from said data items configured to be analysed by said performance parser;
a patient parser, from said set of parsers, in order to output said second output from said data items configured to be analysed by said performance parser);
a Ground Truth Module, in cooperation with said performance parser, establishing Ground Truth data for each cohort, in order to obtain metrics per cohort per data item to be fed to the training module; and
said analysis engine comparing said established ground truth per data item with its own analysed output per data item in order to determine updateable weights based on agreement between said ground truth and said analysed output.

In at least an embodiment, said analysis engine cooperating with:
a ranking module to organize output of said analysis engine in correlation with said first output and said second output; and
said output module configured to generates a mapping between each considered artificially intelligent system and its corresponding trust score.

In at least an embodiment, said system comprising:
said data input module configured to gather data items, including ground truth predictions, per data item, made by one or more of the existing artificially intelligent systems, first output, and said second output; and
said performance parser configured to use said gathered data items, their ground truth predictions, in order to feed to a Training Module which trains internal weights for said analysis engine.

In at least an embodiment, a training module is active only when a ranking module is inactive.

### BRIEF DESCRIPTION OF THE ACCOMPANYING DRAWINGS

This invention will now be described in relation to the accompanying drawing, in which:

FIG. 1 illustrates a schematic block diagram of the system of this invention;

FIG. 2 illustrates a representation of the system of this invention;

FIG. 3 illustrates a diagram illustrating the determination of efficacy;

FIG. 4 illustrates a schematic block diagram for the training module used by this system and method;

FIG. 5 illustrates a schematic block diagram for the ranking module used by this system and method;

FIG. 6 illustrates a schematic block diagram for a first embodiment relating to interaction of the training module, of FIG. 4, and the ranking module, of FIG. 5, used by this system and method; and

FIG. 7 illustrates a schematic block diagram for a second embodiment relating to interaction of the training module, of FIG. 4, and the ranking module, of FIG. 5, used by this system and method.

### DETAILED DESCRIPTION

According to this invention, there are provided systems and methods for selection of priority-wise artificially intelligent mechanisms per one or more characteristics.

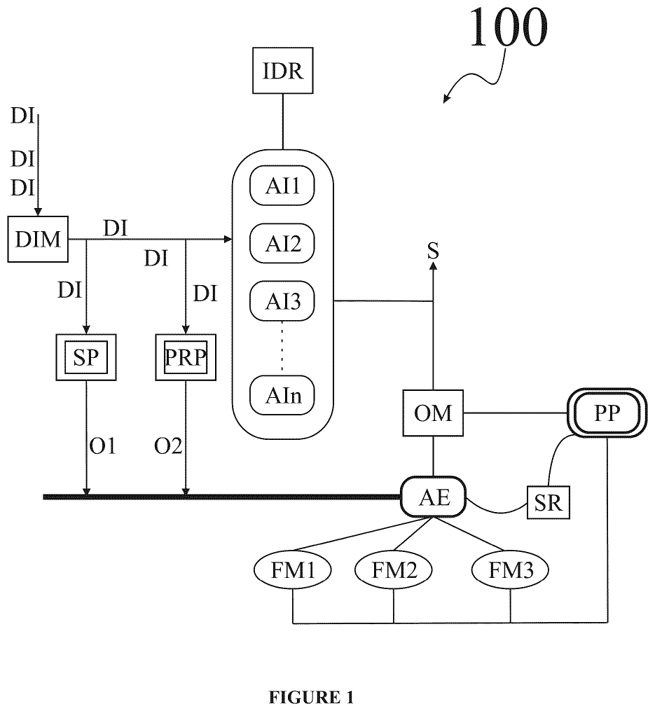

FIG. 1 illustrates a schematic block diagram of the system of this invention.

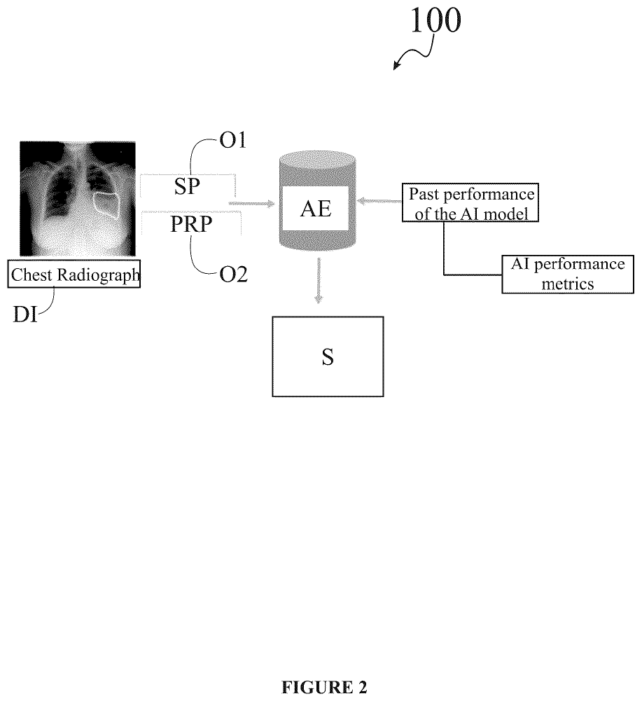

FIG. 2 illustrates a representation of the system of this invention.

In at least an embodiment of the present invention, a data input module (DIM) is configured to receive images, as data input/data items (DI), through this module (DIM). Data items (DI), in at least the form of medical images (with associated metadata), is obtained and passed to further modules of this system and method.

In at least an embodiment of the present invention, an identifier (IDR) is configured to identify an artificially intelligent system (AI1, AI2, AI3, . . . , AIn) being used for determination of efficacy. Each of the data items (DI), from the data input module (DIM), is processed by one or more identified artificially intelligent systems (AI1, AI2, AI3, . . . , AIn).

In at least an embodiment, of the present invention, a scan parser (SP) is configured to parse, and output, scan characteristics, from the data input/data items (DI). This is a first output (scan characteristics) (O1).

In at least an embodiment, of the present invention, a patient parser (PRP) is configured to parse, and output, patient characteristics, from the data input/data items (DI). This is a second output (patient characteristics) (O2).

In at least an embodiment, of the present invention, an analysis engine (AE) is configured to receive at least a first output (i.e. scan characteristics) and/or at least a second output (i.e. patient characteristics) and is further configured to receive feedback signal from a feedback model (FM1, FM2, FM3). An output module (OM), associated with the analysis engine (AE), provides a selection (S), or a choice therefor, of a priority-wise-ranked artificially intelligent system, from the plurality of artificially intelligent systems (AI1, AI2, AI3, . . . , AIn), used by this system and method. The priority-wise ranked output ensures that the selected artificially intelligent system (AI1, AI2, AI3, . . . , AIn) is one of the more efficient/accurate systems for a given set of data items. In other words, the priority-wise ranked output ensures that the selected artificially intelligent system (AI1, AI2, AI3, . . . , AIn) is the most suitable system per parsed scan characteristic (O1) and/or per parsed patient characteristic (O2).

In at least an embodiment, of the analysis engine (AE), there is provided a first feedback module (FM1), per artificially intelligent system (AI1, AI2, AI3, . . . , AIn) such that a feedback relating to accuracy of analysis, correlative to performance metrics of the selected artificially intelligent system, is recorded and fed back to the engine (AE). This records historical data per output. This first feedback module (FM1) may be configured to parse, and record, across data items, one or more of the following parameters:
sensitivity;
specificity;
AUROC;
custom metric.

In at least an embodiment, of the analysis engine (AE), there is provided a second feedback module (FM2), per artificially intelligent system (AI1, AI2, AI3, . . . , AIn) such that a feedback correlative to first output (scan characteristics) of the selected artificially intelligent system, is recorded and fed back to the engine (AE). This records scan data/scan metadata per output. This second feedback module (FM2) may be configured to parse, and record, across data items, one or more of the following parameters:
modality;
scanner manufacturer;
scanner model.

In at least an embodiment, of the analysis engine (AE), there is provided a third feedback module (FM3), per artificially intelligent system (AI1, AI2, AI3, . . . , AIn) such that a feedback correlative to second output (patient characteristics) of the selected artificially intelligent system, is recorded and fed back to the engine (AE). This records patient data/patient metadata per output. This third feedback module (FM3) may be configured to parse, and record, across data items, one or more of the following parameters:
age;
sex;
others.

In at least an embodiment, of the analysis engine (AE), a cohort-based vectorization module is configured to determine, and form, cohort-based data sets and to determine, and form, cohort-based characteristics. In at least an embodiment, of the analysis engine (AE), a sorter (SR) sorts data items (DI) into cohorts according to sorting rules defined in a sorting rule engine. These rules may relate to data/metadata. Accordingly, the scans are divided into cohorts, which are scans that have at least one characteristic in common with a current scan. For example, for n characteristics, cohort 1 could be view position (e.g., chest AP, chest PA, lateral), cohort 2 could be sex (e.g. M/F), cohort 3 could be the age group (i.e. 0-18, 18-35, 35-60, 60+), and so on.

In at least an embodiment, of the analysis engine (AE), a performance processor (PP) uses one or more data items (DI), with specific metadata, as sorted by the sorter (SR), and computes a performance function for all scans belonging to individual cohorts and intersections of two or more cohorts:
x₁: cohort 1
x₂: cohort 2
x₃: cohort 3 . . .
xₙ: cohort n
xₙ₊₁: intersection of cohort 1 and 2
xᵢⱼ: intersection of cohort i and j . . .
xₖ: intersection of cohort 1, 2, 3, . . . , n

In at least an embodiment, of the analysis engine (AE), a dataset comprising various data items (DI) is divided, by the sorter (SR), into cohorts, as taught above, based on patient and scan characteristics.

Typically, ‘cohorts’ are defined as sub-datasets where the data has at least one characteristic in common. As an implication of the definition, data with multiple common characteristics can be grouped into a single, unified cohort. For example: If the data characteristics are gender (Male, Female), age group (0-18, 18+), view position (AP and PA), and scanner type (Scanner A, Scanner B), the scans will be grouped into cohorts in the following manner:
Cohort 1: Data where the patient is Male
Cohort 2: Data where the patient is Female
Cohort 3: Data where patient age is 0-18
Cohort 4: Data where patient age is above 18 years
Cohort 5: Data where scan view position is AP
Cohort 6: Data where scan view position is PA
Cohort 7: Data where scan is acquired with Scanner A
Cohort 8: Data where scan is acquired with Scanner B

In addition to these cohorts, the intersection of multiple characteristics is also considered. This is represented as follows:

Cohort 9: Data where the patient is Male AND below 18 years of age AND scanner view position is AP AND scan is acquired with Scanner A.

Although the combinations of cohorts are exhaustive, they are not being listed, as an addition of a single characteristic would change the number significantly.

It is crucial to note the importance of using cohorts for the data processing stage. These cohorts accurately capture the effect of individual patient and scan characteristics on the data items. Moreover, they also capture the interaction of these characteristics amongst themselves, e.g., if an AI system is performing sub-optimally on males in the 18+ age group, this information will not be missed by the cohorts' system.

In at least an embodiment, of the analysis engine (AE), a feature processor is configured in order to extract features from the cohorts, upon which a performance function is used vide the performance processor (PP). This performance function could be one or a combination of metrics, such as Sensitivity or Specificity or AUROC or F1 Score or a custom metric. A performance function is applied to each cohort to obtain its feature. A set of such features over all cohorts in the data point will form the feature vector X.

For example, consider a data point with the following characteristics—Male, above 18 years of age, AP view position, and Scanner A. A performance function will be applied to each cohort in the data point, i.e., Male, Male AND above 18 years, Male AND AP view, etc., as follows:

$$x 1 = performanceFunction 1 ( Male patients in the dataset )$$

$$x 2 = performanceFunction 2 ( Patients with age above 18 years in the dataset )$$

$$x 3 = performanceFunction 3 ( Patients with scans acquired in AP view in the \\ dataset )$$

$$.$$

$$.$$

$$.$$

$$xn = performanceFunction N ( Some combination of characteristics in the \\ dataset )$$

Typically, vector X, is developed, for each scan where, shape of vector is k*N

The feature vector X will be an array of each feature as follows—

$$X = [ x 1 , x 2 , x 3 , … , xn ]$$

Based on this implementation, data points with the same patient and scan characteristics will have the same feature vectors. This is particularly important as the performance of the AI system over all AI predictions for all patient and scan characteristics is captured. Hence, given there are “k” scans with “n” features, the feature set will have the shape of (k×n).

In at least an embodiment, of the analysis engine (AE), the performance processor (PP) is configured to initialise with its baseline being ground truth. In order to train the model, of this invention, in order to be able to compute priorities, for determination of priority-wise selection, a ground truth has to be set. The ground truth is set based on feedback (whether manual, or machine-fed) where the ground truth is provided. If the prediction, of a particular artificially intelligent system (AI1, AI2, AI3, . . . , AIn), matches the feedback, the ground truth Trust Score is set as 1. In case the prediction, of a particular artificially intelligent system (AI1, AI2, AI3, . . . , AIn), does not match the feedback, the ground truth Trust Score is set as 0. This method of setting the ground truth captures trust in a particular artificially intelligent system (AI1, AI2, AI3, . . . , AIn) over the entire data collection period. Therefore, the output feature set will have the shape of (k×1).

As an intialiser, the ground truth for a weighted output, of the output module (OM) (denoted by y) prediction can be a value between 0-100 and is defined as a measure of agreement or disagreement between the artificially intelligent system (AI1, AI2, AI3, . . . , AIn) and a person using the artificially intelligent system (AI1, AI2, AI3, . . . , AIn) for a given one or more data items (DI).

Typically, this performance function, vide the performance processor (PP), is applied to each cohort.

Typically, this performance function, vide the performance processor (PP), could be one or a combination of common machine learning classification metrics, including but not limited to sensitivity, specificity, AUROC, F1 score or a custom metric; as recorded/determined by the first feedback module (FM1). The trust score will then be calculated as:

$$\hat{y} = f ( [ {x}_{1} , {x}_{2} , … , {x}_{k} ] ) ,$$

where,
f:R^{k}→R is a regression or classification model,
X=[x₁, x₂, . . . , xₗ] is the input vector,
and ŷ is the predicted trust score of the scan.
The trust score will be optimized using a loss function g:R×R→R such that g(ŷ, y) is minimized.

In at least an embodiment, of the analysis engine (AE), the performance processor (PP) is configured to determine how weight affect loss function.

Based on this dataset, the Trust Score (represented as ŷ) will then be calculated as the following:

$$\hat{y} = f ( [ {x}_{1} , {x}_{2} , … , {x}_{n} ] ) ,$$

where f:R^{k}→R is a regression or classification model, such as linear regression or logistic regression or bayesian regression or Support Vector Machines (SVM) or K-nearest neighbors (KNN) or Gaussian Naive Bayes or Multinomial Naive Bayes or Complement Naive Bayes or Bernoulli Naive Bayes or Decision Trees or Random Forests or Gradient Boosting or Neural Networks, which will be fitted using the input vector X==[x₁, x₂, . . . , xₙ], and output Q, which is the predicted trust score of the scan. This calculation is done by assigning importance, in the form of weights, to each feature. These weights can be randomly or manually initialized. During the course of training, these weights will be optimized using a loss function, g:R×R→R, such as Binary Cross-Entropy or Hinge or Squared Hinge or Sigmoid Cross-Entropy or Kullback-Leibler (KL) Divergence, Focal Loss or Weighted Cross-Entropy Loss or Squared Error Loss or Huber Loss or Area Under Receiver Operating Characteristic (AUROC) curve Loss or Contrastive Divergence Loss or Center Loss or Gaussian Mixture Loss or Huber Loss or some custom loss function such that g(ŷ, y) is minimized.

Once the parameters of the machine learning model are determined, it elucidates the relative importance of the different scan and/or patient characteristics. Using that information, the system and method of this invention is able to provide an explanation for low, medium, or high trust scores, for example, if the artificially intelligent system has previously not performed well for a particular age group.

In preferred embodiments, priority-wise-ranked selection of artificially intelligent systems is determined by applying threshold values, based on a rule engine. Once the parameters of the regression or classification model are determined, it elucidates relative importance of different scan and/or patient characteristics. Using that information, the system and method, of this invention, is able to provide an explanation for the priority-wise-ranked selection of artificially intelligent systems per one or more scan characteristic/s, per one or more patient characteristic/s, or the like one or more characteristic.

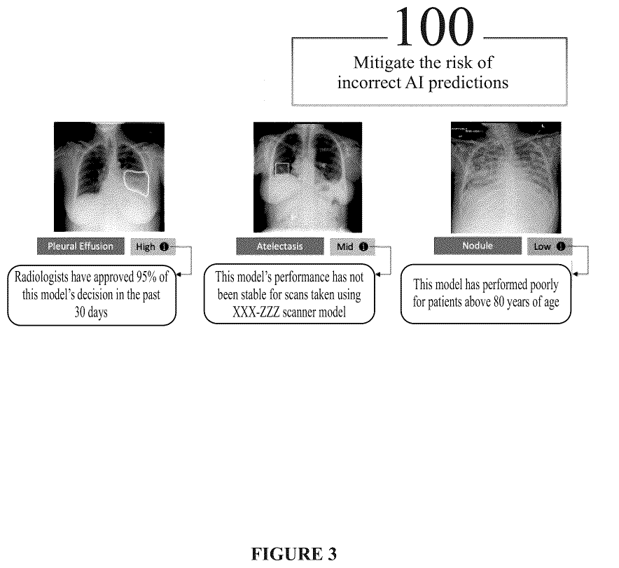

FIG. 3 illustrates a diagram illustrating the determination of efficacy.

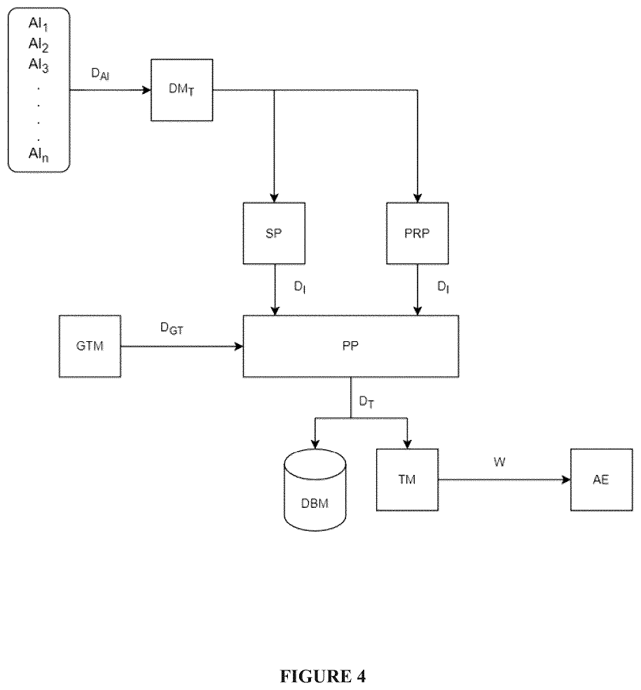

FIG. 4 illustrates a schematic block diagram for the training module used by this system and method.

In the Training Module, one or more of the existing artificially intelligent systems (AI1, AI2, AI3, . . . , AIn) generate AI data (DAI) for scans previously annotated by users of these systems. These predictions, along with the data of the input files to these artificially intelligent systems, are organized in the Training Data Module (DMT).

The Scan Parser (SP) and the Patient Parser (PRP) extract respective scan characteristics and patient characteristics from input files and generate input data (DI), which is analyzed by the performance parser (PP).

The performance parser (PP) generates metrics such as Sensitivity, Specificity, AUROC, and custom metrics for each cohort using the Ground Truth data (DGT) generated by the Ground Truth Module (GTM). The metrics per cohort per scan generated by the Performance Parser (PP) serve as the Training Data (DT) and are passed to the Database Module (DBM) and the Training Module (TM). The algorithm undergoes training by comparing Ground Truth data (DGT) with the predictions from the existing artificially intelligent systems (AI1, AI2, AI3, . . . , AIn) in the training module (TM). During this process, the algorithm's internal weights (W) are updated based on the agreement between the ground truth and the AI predictions. Following training, the algorithm with updated internal weights (W), is utilized within the Analysis Engine (AE).

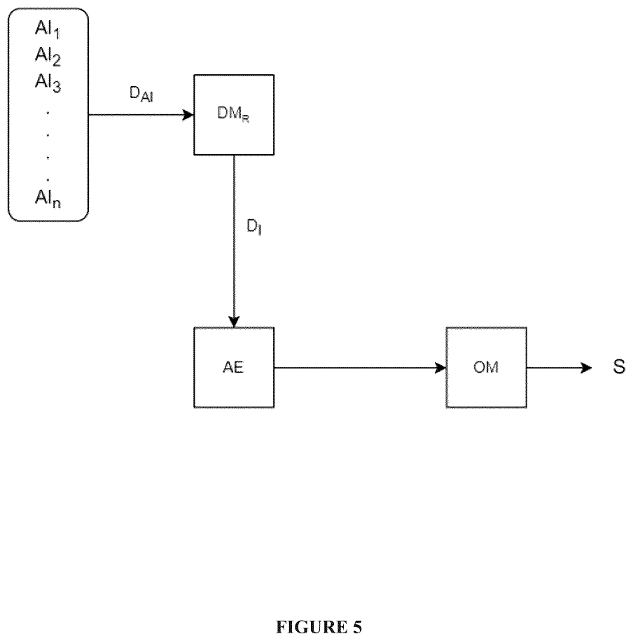

FIG. 5 illustrates a schematic block diagram for the ranking module used by this system and method.

Data generated (DAI) by one or more of the existing artificially intelligent systems (AI1, AI2, AI3, . . . , AIn) is sent to the Ranking Data Module (DMR). This module organizes the predictions and associated scan and patient characteristics, forming the input data (DI). DI is then transmitted to the Analysis Engine (AE), where the trust scores for one or more of the existing artificially intelligent systems (AI1, AI2, AI3, . . . , AIn) are evaluated. The Output Module (OM) then generates a mapping between each considered artificially intelligent system and its corresponding trust score. The selection module (S) orchestrates the ranking of these artificially intelligent systems based on their trust scores, providing a streamlined representation of their reliability.

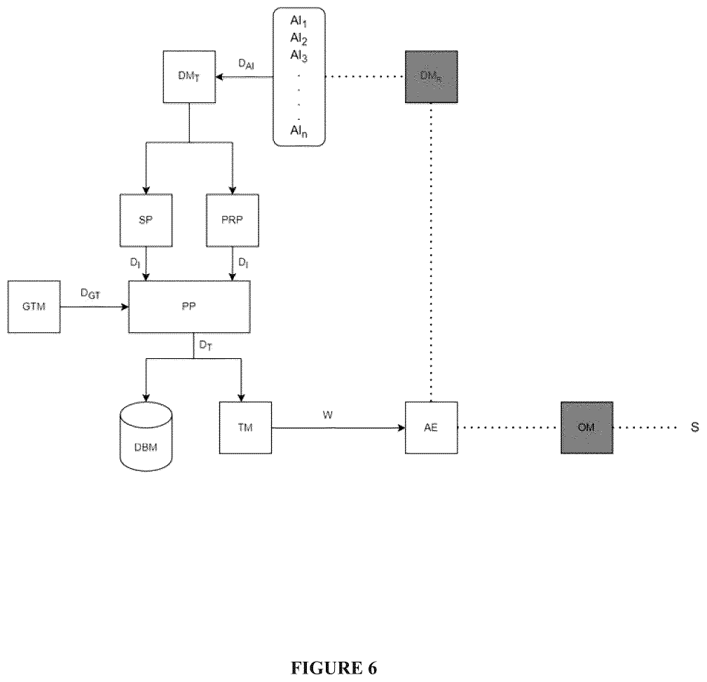

FIG. 6 illustrates a schematic block diagram for a first embodiment relating to interaction of the training module, of FIG. 4, and the ranking module, of FIG. 5, used by this system and method.

During the data collection phase, the system gathers all data including ground truth, predictions made by one or more of the existing artificially intelligent systems (AI1, AI2, AI3, . . . , AIn), scan characteristics, and patient characteristics. This data is then utilized by the Performance Parser (PP), followed by the Training Module in which the internal weights (W) of the Trust Score algorithm are updated. In this phase, the Training Module (TM) takes precedence and is active while the Ranking Data Module (DMR) is inactive. This is because the ranking of artificially intelligent systems cannot be determined during the data collection phase. The updated weights are stored by the Analysis Engine (AE).

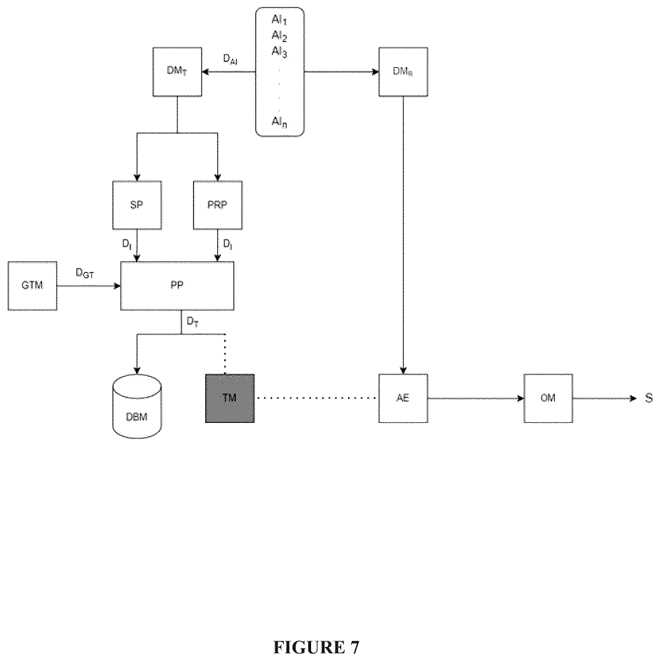

FIG. 7 illustrates a schematic block diagram for a second embodiment relating to interaction of the training module, of FIG. 4, and the ranking module, of FIG. 5, used by this system and method.

After the data collection phase, the Database Module (DBM) continues to store the data generated by the Performance Parser (PP) but the Training Module (TM) becomes inactive as the algorithm will have optimized its internal weights (W) using the data collected during the data collection phase. The Ranking Data Module (DMR) and Analysis Engine (AE) become active within the system process flow and the algorithm becomes eligible to evaluate and rank one or more of the existing artificially intelligent systems (AI1, AI2, AI3, . . . , AIn).

With the use of this system and method, the output provides radiologists, using one of many artificially intelligent mechanisms, confidence while using the AI mechanism. The output also comprises an explanation in correlation with efficacy. This enables users to:
1) Take an informed decision to agree or disagree with AI predictions based on its past performance.
2) Determine the weight, significance, and importance to be assigned to the AI output.
3) Understand the model better and predict its future behavior.

According to a first non-limiting exemplary embodiment, the system and method, of this invention was used to test performance of Tuberculosis-AI model by Vendor 1 (V1) on different cohorts.

TABLE 1

| AI | | Age | View | Scanner | |
|---|---|---|---|---|---|
| Model | Gender | Group | Position | Type | AUROC |
| TB | M | 0-18 | AP | S1 | 0.89 |
| | M | 18-35 | AP | S1 | 0.91 |
| | F | 35-60 | AP | S2 | 0.94 |
| | F | 60+ | AP | S2 | 0.9 |
| | M | 0-18 | AP | S1 | 0.88 |
| | M | 35-60 | AP | S1 | 0.9 |
| | F | 18-35 | AP | S2 | 0.93 |
| | F | 35-60 | AP | S2 | 0.94 |

This table 1 illustrates past performances, quantified by an AUROC score, for an AI system designed to detect Tuberculosis in Chest X-rays. The performance of the AI system is consistent over various cohorts such as gender, age group, view position, and scanner type. Therefore, for a scan parsed through the TB detection AI system, a user can accept the AI prediction without much scrutiny.

According to a second non-limiting exemplary embodiment, the system and method, of this invention was used to test performance of Tuberculosis-AI model by Vendor 1 (V2) on different cohorts.

TABLE 2

| AI | | Age | View | Scanner | |
|---|---|---|---|---|---|
| Model | Gender | Group | Position | Type | AUROC |
| TB | M | 0-18 | AP | S1 | 0.593 |
| | M | 0-18 | PA | S1 | 0.61 |
| | F | 0-18 | AP | S2 | 0.55 |
| | F | 0-18 | PA | S2 | 0.53 |
| | M | 18-35 | AP | S1 | 0.92 |
| | M | 35-60 | PA | S1 | 0.94 |
| | F | 60+ | AP | S2 | 0.76 |

This table 2 illustrates past performances, quantified by the AUROC score, for an AI system designed to detect Tuberculosis in Chest X-rays. Notably, the AI system exhibits subpar performance for scans of patients in the 0-18 age group. Conversely, it demonstrates moderate performance for patients aged 60 and above and excellent performance for those in the 18-35 and 35-60 age groups. Here, the user would not have to scrutinize the AI prediction much if it belongs to a patient in the 18-35 or 35-60 age groups. However, they would have to focus more on the AI predictions for the scans for the patients in the 0-18 and 60+ age groups.

In the case of a chest X-ray from a patient in the 0-18 age group, the user may confidently rely on the prediction of the AI system from V1, given its robust performance. Conversely, when evaluating a PA scan acquired on scanner Si from a male (M) patient of age group 35-60, the user may opt for V2, given its superior AUROC score.

According to a third non-limiting exemplary embodiment, the system and method, of this invention was used to test performance of AI model that detect abnormalities in lateral Chest X-rays on different cohorts.

TABLE 3

| AI | | Age | View | | |
|---|---|---|---|---|---|
| Model | Gender | Group | Position | Vendor | AUROC |
| Abnormal | M | 0-18 | Lateral | V1 | 0.63 |
| | M | 18-35 | Lateral | V1 | 0.73 |
| | F | 0-18 | Lateral | V1 | 0.64 |
| | F | 18-35 | Lateral | V1 | 0.75 |
| | M | 0-18 | Lateral | V2 | 0.73 |
| | M | 18-35 | Lateral | V2 | 0.89 |
| | F | 0-18 | Lateral | V2 | 0.71 |
| | F | 18-35 | Lateral | V2 | 0.91 |

This table 3 illustrates past performances of an AI system, quantified by the AUROC score, for an AI system designed to detect abnormalities in Lateral chest X-rays. The performance of V1 is consistently inferior across all age groups compared to V2. Notably, within the V1 subset, the performance of the model is subpar for the patients in the 0-18 age group. This could be due to insufficient data during model training specifically for age group 0-18, or due to suboptimal training parameters. These findings underscore the lack of reliability on V1 for detection of abnormalities from lateral Chest X-rays. Based on the past performance of AI models by V1 and V2, the user may rely on the predictions of V2 as compared to predictions of V1.

The TECHNICAL ADVANCEMENT of this invention lies in systems and methods for selecting a priority-wise artificially-intelligent system based on one or more characteristics. This selection endorses trust values in various artificially-intelligent systems.

The following discussion is intended to provide a brief, general description of Suitable computing environments in which the system and method may be implemented. Although not required, the disclosed embodiments will be described in the general context of computer-executable instructions, such as program modules, being executed by a single computer. In most instances, a “module’ constitutes a software application.

Generally, program modules include, but are not limited to routines, Subroutines, software applications, programs, objects, components, data structures, etc., that perform particular tasks or implement particular abstract data types and instructions. Moreover, those skilled in the art will appreciate that the disclosed method and system may be practiced with other computer system configurations, such as, for example, hand-held devices, multi-processor systems, data networks, microprocessor-based or programmable consumer electronics, networked PCs, minicomputers, mainframe computers, servers, and the like.

For the purposes of this specification, term ‘module’, as utilized herein, may refer to a collection of routines and data structures that perform a particular task or implements a particular abstract data type. Modules may be composed of two parts: an interface, which lists the constants, data types, variable, and routines that can be accessed by other modules or routines, and an implementation, which is typically private (accessible only to that module) and which includes source code that actually implements the routines in the module. The term module may also simply refer to an application, such as a computer program designed to assist in the performance of a specific task, such as word processing, accounting, inventory management, etc.

The interface, which is preferably a graphical user interface (GUI), can serve to receive inputs for engagement with other modules as also to display results, whereupon a user may supply additional inputs or terminate a particular session.

While this detailed description has disclosed certain specific embodiments for illustrative purposes, various modifications will be apparent to those skilled in the art which do not constitute departures from the spirit and scope of the invention as defined in the following claims, and it is to be distinctly understood that the foregoing descriptive matter is to be interpreted merely as illustrative of the invention and not as a limitation.

1. A computer-implemented method for selection of a priority-wise artificially intelligent mechanism from a plurality of artificially intelligent systems per one or more characteristics, the method comprising:
receiving, by a data input module, medical images as data items including associated metadata;
identifying, by an identifier, at least one artificially intelligent system used for determination of efficacy;
processing, by the identified artificially intelligent system, each of the data items to generate AI outputs;
parsing, by a set of parsers, the data items to output,
a first output comprising scan characteristics obtained by a scan parser, and
a second output comprising patient characteristics obtained by a patient parser;
analyzing, by a cohort-based vectorization module within an analysis engine, the first output and/or the second output together with feedback signals from a feedback model including,
a first feedback module recording accuracy metrics correlated to performance metrics of each artificially intelligent system,
a second feedback module recording scan-characteristic-correlated feedback, and
a third feedback module recording patient-characteristic-correlated feedback;
sorting, by a sorter within the analysis engine, the data items into cohorts based on sorting rules defined in a sorting rule engine and correlated to the scan characteristics and/or patient characteristics;
computing, by a performance processor, performance metrics for individual cohorts and intersections of cohorts, wherein the performance metrics are correlated to one or more of sensitivity, specificity, AUROC, F1-score, and a custom metric;
generating, by a feature processor, feature vectors from the performance metrics, wherein each feature vector corresponds to one of the data items and has a shape defined by a first set of the data items and a second set of features;
setting, by a ground truth module, ground truth values for each cohort based on manual or machine-fed mechanisms;
comparing, by the ground truth module, analyzed outputs to the ground truth to update internal weights of the analysis engine;
calculating, by the analysis engine, trust scores for each artificially intelligent system using a regression or classification model with the feature vectors as inputs, wherein the trust scores are optimized by minimizing a defined loss function; and
serving, by an output module, a selection of a priority-wise-ranked artificially intelligent system from the plurality of artificially intelligent systems, wherein the selection is specific to the parsed scan characteristics and/or parsed patient characteristics so as to improve the reliability and adoption of the selected artificially intelligent system in medical image analysis.

2. The method of claim 1, wherein each feature vector generated by the feature processor is formed by collating features per cohort, the feature vector having a shape defined by a first set of data items and a second set of features, such that the feature set has a dimensionality of (the first set of data items×the second set of features).

3. The method of claim 1, wherein the priority-wise ranking is determined by applying a threshold-based rule engine to the calculated trust scores, wherein the thresholds are dynamically updated based on the relative importance of the scan characteristics and/or patient characteristics as determined by the machine learning model.

4. The method as claimed in claim 1, wherein the analyzing includes a cohort-based vectorization analysis configured to determine, and form, cohort-based data sets to determine, and form, cohort-based characteristics, wherein the analysis includes,
sorting data items, basis its data and/or its metadata, into cohorts according to sorting rules, correlative to the first output and/or the second output, defined in a sorting rule engine,
using, by a performance processor, one or more of the data items, with specific metadata, as sorted by the sorter, to compute a performance function for all data items belonging to individual cohorts and intersections of two or more cohorts, and
extracting features from the cohorts, upon which a performance function is used by the performance processor, wherein the performance function is correlated to one or more metrics relating to the first output, wherein the performance function is applied to each cohort to obtain its feature, wherein the feature processor is configured to collate a set of obtained features, per cohort, to form a feature vector X, for each data item, and each vector is shaped by a first set of data items and a second set of features.

5. The method as claimed in claim 1, wherein the analysis engine includes a cohort-based vectorization module configured to perform,
receiving the first output and/or the second output and a feedback signal from a feedback model,
computing cohorts based on sorting rules defined in a sorting rule engine, followed by grouping data items with common characteristics,
processing cohorts, using a performance processor, to compute performance metrics for individual cohorts and their intersections,
developing feature vectors based on performance metrics, for data items, sharing common characteristics,
setting a ground truth for training artificially intelligent system using manual mechanisms or machine-fed mechanisms,
calculating trust scores for data items using regression or classification models with weighted features, minimizing a loss function, and
determining prioritization of artificially intelligent system based on threshold values and a rule engine considering the relative importance of characteristics identified by machine learning models.

6. The method as claimed in claim 1, wherein the analyzing is performed in cooperation with,
generating data, from the first feedback module, correlative to the data items,
outputting the first output from the data items configured to be analyzed by the performance parser,
outputting the second output from the data items configured to be analyzed by the performance parser,
establishing Ground Truth data for each cohort, to obtain metrics per cohort per data item to be fed to a training module, and
comparing the established ground truth per data item with its own analyzed output per data item to determine updateable weights based on agreement between the ground truth and the analyzed output.

7. The method as claimed in claim 1, further comprising:
gathering data items, including ground truth predictions, per data item, made by one or more of the existing artificially intelligent systems, first output, and the second output; and
using the gathered data items, their ground truth predictions, to feed to a Training Module which trains internal weights for the analysis engine.

8. A system for selection of a priority-wise artificially intelligent mechanism from a plurality of artificially intelligent systems per one or more characteristics, the system comprising:
a data input module configured to receive medical images as data items comprising associated metadata;
an identifier configured to identify at least one artificially intelligent system used for determination of efficacy, wherein each of the data items is processed by one or more of the identified artificially intelligent systems;
a set of parsers including,
a scan parser configured to parse and output scan characteristics as a first output, and
a patient parser configured to parse and output patient characteristics as a second output;
a feedback model including,
a first feedback module to record accuracy metrics correlated to performance metrics of each artificially intelligent system,
a second feedback module to record scan-characteristic-correlated feedback, and
a third feedback module to record patient-characteristic-correlated feedback;
an analysis engine including a cohort-based vectorization module configured to,
receive the first output, the second output, and the feedback signals,
sort data items into cohorts using a sorter based on sorting rules defined in a sorting rule engine,
compute performance metrics for each cohort and intersection of cohorts using a performance processor,
generate feature vectors from the computed performance metrics using a feature processor, wherein each vector is shaped by a first set of data items and a second set of features,
compare analyzed outputs with ground truth values from a ground truth module to update internal weights of the analysis engine, and
calculate trust scores for each artificially intelligent system using a regression or classification model with the feature vectors as inputs, the trust scores being optimized by minimizing a defined loss function;
an output module cooperating with the analysis engine to map each artificially intelligent system to its corresponding trust score and to serve a selection of a priority-wise-ranked artificially intelligent system from the plurality, wherein the selection is specific to the parsed scan characteristics and/or parsed patient characteristics; and
a ranking module configured to organize the output of the analysis engine and present the ranked selection for use in medical image interpretation.

9. The system of claim 8, wherein the feature processor is configured to generate feature vectors for each data item by collating features per cohort, and wherein each feature vector has a shape defined by a first set of data items and a second set of features.

10. The system of claim 8, wherein the ranking module applies a threshold-based rule engine to the calculated trust scores to generate the priority-wise ranking, and wherein the thresholds are dynamically updated based on the relative importance of the scan characteristics and/or patient characteristics as determined by the machine learning model.

11. The system as claimed in claim 8, wherein the analysis engine is a cohort-based vectorization module configured to determine, and form, cohort-based data sets to determine, and form, cohort-based characteristics, and wherein the analysis engine includes,
a sorter configured to sort data items, based on data of the sorter and/or its metadata, into cohorts according to sorting rules, correlative to the first output and/or the second output, defined in a sorting rule engine,
a performance processor configured to use one or more data items, with specific metadata, as sorted by the sorter, to compute a performance function for all data items belonging to individual cohorts and intersections of two or more cohorts, and
a feature processor configured to extract features from the cohorts, upon which a performance function is used by the performance processor, wherein the performance function correlates to one or more metrics relating to the first output, wherein the performance function is applied to each cohort to obtain its feature, wherein the feature processor is configured to collate a set of obtained features, per cohort, to form a feature vector X, for each data item, and wherein each vector is shaped by a first set of data items and a second set of features.

12. The system as claimed in claim 8, wherein the analysis engine is a cohort-based vectorization module configured to perform,
receiving the first output and/or the second output and a feedback signal from a feedback model,
computing cohorts based on sorting rules defined in a sorting rule engine, followed by grouping data items with common characteristics,
processing cohorts, using a performance processor, to compute performance metrics for individual cohorts and their intersections,
developing feature vectors based on performance metrics, for data items, sharing common characteristics,
setting a ground truth for training artificially intelligent system using manual mechanisms or machine-fed mechanisms,
calculating trust scores for data items using regression or classification models with weighted features, minimizing a loss function, and
determining prioritization of artificially intelligent system based on threshold values and a rule engine considering the relative importance of characteristics identified by machine learning models.

13. The system as claimed in claim 8, wherein the analysis engine is configured to cooperate with,
a training module configured to generate data, from the first feedback module, correlative to the data items,
a scan parser, from the set of parsers, configured to output the first output from the data items configured to be analyzed by the performance parser,
a patient parser, from the set of parsers, configured to output the second output from the data items configured to be analyzed by the performance parser, and
a Ground Truth Module, in cooperation with the performance parser, configured to establish Ground Truth data for each cohort, to obtain metrics per cohort per data item to be fed to the training module, wherein the analysis engine is configured to compare the established ground truth per data item with its own analyzed output per data item to determine updateable weights based on agreement between the ground truth and the analyzed output.
